(19) United States
(12) Patent Application Publication
(10) Pub. No.: US 2008/0115234 A1
(43) Pub. Date: May 15, 2008

(54) Method for utilization of domestic ruminants with mycobacterium avium subspecies paratuberculosis disease to assess possible therapeutic modalities

(76) Inventor: Gilles Reza George Monif, Bellevue, NE (US)

Correspondence Address: Gilles R.G. Monif, 17121 Lakewood Drive, Bellevue, NE, 68123-3954, US

(21) Appl. No.: 11/521,200

(22) Filed: Sep. 14, 2006

(51) Int. Cl. A01K 67/027; C12Q 1/68

(52) U.S. Cl. 800/14; 800/15; 800/16; 435/6; 435/7.92

(57) ABSTRACT

The invention describes a concrete schema which allow domesticated ruminant with disease due to Mycobacterium avium subspecies paratuberculosis to serve as an animal model system in order to assess potential systemic and mucosal effects achieved by attempted therapeutic interventions. A key component of this schema is the recognition of the significance of a positive precipitantion band on agar gel immunodiffusion test in terms of the underlying histopathology and subsequent ability to use such data in evaluation necropsy derived data. A second component is the sequential use of newly developed nesting PCR technology and their abilities to identify (and where indicated quantitate) Map DNA present in feces, blood and milk.

### BACKGROUND OF THE INVENTION

(1) Field of Invention

The method involves the sequential applications of newly developed technology which allows utilization of ruminants infected with Mycobacterium avium subspecies paratuberculosis to serve as model systems for the evaluation of therapeutic interventions

(2) Description of the Prior Art

Map infection of domestic and non-domesticated ruminants is global in its distribution. Map is the recognized inducer of a chronic granulomatous gastrointestinal disease (Johne's disease) in dairy cattle. The estimated annual economic loss to the dairy industry approaches $1.5-2 billion. While estimates of herd prevalence are comparatively low, within large herds, up to 40% of cows my be infected. Within herds with high infection rates, removal for slaughter of all test-positive cows would create an economically unfeasible situation.

The national herd management response to combat Johne's disease has been to adopt a policy of testing and culling serologically highly positive animals. Standards for sensitivity and specificity of commercial enzyme-linked immunosorbent assays (ELISA) have been largely developed established using fecal culture-positive cattle. The use of fecal-positive cattle and not necropsy confirmed cattle presumes the infallibility of fecal identification which, while the gold diagnostic standard, fecal recovery techniques are not inclusive of all serologically positive infected cows.

Collins et. al. evaluated five antibody detection tests for the diagnosis of bovine paratuberculosis using serum samples from 359 dairy cattle in seven paratuberculosis-free herds and 2,094 dairy cattle in seven Map-infected dairy herds. Both ParaCheck (Biocor, Omaha, Nebr.) and HerdCheck (IDEXX Laboratories Inc. Westbrook, Me.) ELISA tests done in accordance with manufacturers' instruction and interpreted as prescribed by the kit insert, identified less than 29% of fecal culture positive cows. A positive relationship could be shown between the number of mycobacterium in feces and ELISA positivity. With a low number of Map in feces, a mean of 13.3% of infected cows were ELISA positive. At progressively higher fecal culture scores, the mean percentage of positive antibody assays were 27.3% 54.9% and 78.4% respectively.

Map has been isolated from up to 8% in milk of subclinically infected animals. Giese and Ahrens isolated Map from up to 35% of clinically infected animals. The Marshfield Clinic retail milk study found viable Map in 2.8% of milk samples taken directly from grocery stores in three of the nation's five largest dairy states. Map DNA was demonstrated in 64% of the 702 pints of milk sampled. Using nesting PCR, Buergelt and Williams, while showing a general positive correlation between increasing Map ELISA reading and increased probability of detection of Map DNA in milk, demonstrated that cows with initial low positive ELISA, initial suspicious ELISA and negative ELISA reading could potentially have Map DNA detected in their milk.

Crohn's disease is a chronic granulomatous disease affecting about 600.000 individuals in the United States. The interest in herd management emanates from a growing body of circumstantial evidence indicating that Map is the etiological agent for Crohn's disease. Using polymerase chain reaction, Map DNA can be identified in 60-80% of gastrointestinal mucosal specimens taken from individuals with Crohn's disease. Map has been cultured from the breast milk of 2 out of 5 lactating women with Crohn's disease and from the blood of other afflicted individuals.

The strong circumstantial case that Map is the causative agent of Crohn's disease has shifted in therapeutic interventions from animals with unique genetic composition (zoo animals) and uniquely valuable domestic animals to a broader agenda. The current therapy of Crohn's disease involves immunologically knocking out the individual's Th1 immune response which appears to arrest continuing tissue damage. The human body's TH1 immune response is largely responsible for virus and mycobacterium arrestment.

The inventor and collaborators at the University of Florida have developed unique PCR and nesting PCR technology which allows one to identify Map DNA in feces and milk. This technology has meet the standard for certification for fecal identification in three blinded tests of USDA/APHIS. This technology renders data results within three hours.

The gold diagnostic standard for Map infection/disease has been based upon fecal recovery using artificial culture media. The technique is highly specific, but is suboptimal in terms of sensitivity. These techniques using artificial culture media are not inclusive of all serologically positive infected cows. At necropsy, cows have been shown to have infection/disease and yet their fecal cultures failed to grow the organism. Beyond the issue of limited sensitivity, there is the time in which a culture must be observed before being deemed negative. Cultures may become positive as long as six months after inoculation. In very rare instances, cultures have been reputed to become positive between six months and one year

In the past two years, culture on artificial media has been pragmatically replaced by the Bactec MGIT 960 Mycobacterial System. The test involves specialized liquid media, patent sensors. Its advanced fluorometric technology permits use of highly accurate detection of oxygen consumption. Suspected cultures are then tested using standard PCR technology which involves IS900 derived primers. The Bactec system requires purchase of a specifically designed incubator (cost range $39-45,000) as well as antibiotics The time before a culture is deemed negative is 49 days.

### SUMMARY OF THE INVENTION

The present invention is directed towards a sequence of management steps which allows for qualitative and quantitative assessment of organismal presence as well as its secondary influence on organism induced serological parameters in domestic ruminants. The method steps include:
a) selecting an appropriate test animal on he basis of being able to demonstrate the presence of a precipitation band when the animal's serum is tested using agar gel immunodiffusion assay, a significant Map ELISA titer, and the presence of Map DNA in feces and possibly blood and milk. Prior necropsy analysis of 10 dairy cows with Johne's disease has documented that each cow meeting these entry criteria had multibacillus status,
b) if the cow is lactating, PCR analysis to demonstrate presence or absence of Map DNA in milk is added as well as that of blood,
c) three separate measurements are done to establish baseline values (Table 1),
d) samples of all evaluation points (fecal and blood Map DNA determination, milk Map DNA, serum AGID, serum Map ELISA titer) are sequentially done no later than one week apart Table 1),
e) monitored criteria reflective of a therapeutic effect on a systemic level are: primarily AGID, Map ELISA titer, and Map DNA in blood,
f) monitored criteria indicative of a therapeutic effect on a mucosal level are: Map DNA in feces, Map DNA in milk,
g) definitive monitored criteria of a therapeutic effect are Map DNA in feces and milk (if available) and histopathological assessment of the quantity of mycobacteria present compared to previously established necropsy standards.

One aspect of the invention is that it monitors Map induced specific antibodies (AGID and ELISA) and can identify significant changes in these parameters.

Another aspect of the invention is that it qualitatively (inventor's nesting PCR )and quantitatively (real time nesting PCR) can monitor Map DNA changes within fecal and milk which reflect organismal activity within mucosal surfaces.

Another aspect of the invention is that it can provide documentation of quantitative changes in the number of mycobacteria within the target organ (gastro-intestinal tract) which may occur as a consequence of a therapeutic intervention. The number of mycobacteria identified in histological section of cecum and ileum by special stains in a given slide are compared with those standards established in previous necropsied dairy cows meeting the entry criteria. Perception that historically established standards for organismal presence and structural histological characterization of tissue architecture can be effectively used to demonstrate a possible therapeutic response is a key observation as it gives a relative point-by-point comparison.

TABLE 1
Schematic for Utilization of Ruminants (Dairy Cows)
As A Animal Model For Assessing Attempted Therapeutic
Intervents' Effects on Mycobacterium Avium Subspecies Paratuberculosis

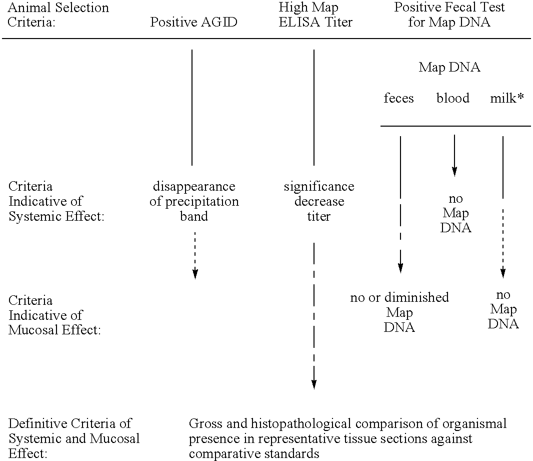

*applicable to lactation diseased ruminants

TABLE 2
Results From a Therapeutic Intervention
of a Holstein Cow With Johne's Disease
(Earlier Example - Not Done with Current Technology)

| | | ELISA | Fecal | nesting PCR | |
|---|---|---|---|---|---|
| Dates | AGID | Titer | Culture | Blood | Milk |

Cow #6142

| | | | | | |
|---|---|---|---|---|---|
| 14/15/01 | negative | 1.8 | n.d. | n.d | n.d. |

Baseline Studies

| | | | | | |
|---|---|---|---|---|---|
| Sep. 10, 2002 | positive | 3.2 | n.d. | negative | n.d. |
| Sep. 24, 2002 | positive | 3.0 | n.d. | negative | positive |

Intervention - Sep. 26, 2002

| | | | | | |
|---|---|---|---|---|---|
| Nov. 19, 2002 | negative | 1.8 | n.d | positive | positive |
| Dec. 10, 2002 | negative | 1.5 | contaminated | positive | positive |
| Dec. 30, 2002 | negative | 2.0 | positive | positive | positive |
| Jan. 21, 2003 | negative | 2.7 | negative | positive | positive |
| Feb. 04, 2003* | negative | 2.3 | negative | positive | negative |

ELISA Titer greater than 2.0 = positive
n.d. = not done
*date of necropsy
**fecal cultures done on solid media

TABLE 3
Comparison of Necropsy on Cow #6142
and a Cow Representative of the 10 AGID Positive Controls
(see attached necropsy reports)

Tables 1 and 2 are a real world demonstration of how the model system produced concrete evidence of a beneficial effect as a consequence of the therapy applied.

I claim:

1: An animal model system using one or more cows with disease due to Mycobacterium avium subspecies paratuberculosis (Map) and related genetic mutants can assesss potential changes induced by diverging therapeutic interventions can be assessed. The steps compromise
a) selection of a diseased cow with one or more precipitation Map bands demonstrable by agar gel immunodiffusion (AGID),
b) selection of a ruminant with a high Map enzyme-linked immunoabsortion (ELISA) titer,
c) selection of one or more cows with Map DNA or mycobacteria demonstrable in its fecal specimens,
d) establishment of mean baseline values based upon three or more sets of tests
e) administration of presumed therapeutic modality/agent/regimen
f) serial (minimal weekly or as deemed statistically appropriate) evaluation of each test animal using AGID positivity or negativity, changes in ELISA titer, presence or absence of Map of Map DNA in fecal samples using direct fecal nested Map PCR, and fecal real time Map PCR testing
g) quantization of Map DNA in feces using real time PCR
h) submission of the cow to necropsy for gross examination of the gastrointestinal tract and its examination using special stains to quantize the number of mycobacterium present on a representative section of cecum and ileum and corresponding lymph nodes.

2: Domestic ruminants are cattle, dairy cows, goats, sheep and buffaloes.

3: The method claims that reduction of ELISA titer to below the optical reading deemed suspicious of Map infection, the loss of AGID positivity, elimination of Map DNA from milk specimens, the reduction of Map DNA in serial fecal specimens as determined by real-time Map PCR, and the reduction in the number of mycobacteria demonstrable with diseased sections of the gastrointestinal tract obtained at necropsy, collectively or in combination assess the degree of therapeutic effectiveness of a given regimen.

4: The method claimed in 1, 2 and 3 can be used to identify drugs potentially effective on Map and genomic mutants of Map.
